(19) United States
(12) Patent Application Publication
(10) Pub. No.: US 2024/0161889 A1
(43) Pub. Date: May 16, 2024

(54) SYSTEMS AND METHODS FOR PROVIDING ACCESS TO ELECTRONIC HEALTH RECORDS USING A VIRTUAL HEALTH WALLET

(71) Applicant: Change Healthcare Holdings, LLC, Nashville, TN (US)

(72) Inventors: Andras L. Ferenczi, Scottsdale, AZ (US); Srilekha Akula, Fremont, CA (US)

(21) Appl. No.: 18/055,578

(22) Filed: Nov. 15, 2022

(51) Int. Cl. G16H 10/65

(52) U.S. Cl. CPC G16H 10/65

(57) ABSTRACT

Systems and methods for using a virtual health wallet are provided. The wallet is created for a patient by an SSI service and is associated with a DID that is associated to the patient along with a public and private key pair. The DID of the patient is stored on a distributed ledger. The wallet is stored on a cloud-based endpoint. When a record is created for a patient by a provider, the record is stored in the wallet at the endpoint, and a DID of the record is stored in the ledger along with an address of the endpoint. Later the user may share the DID of the record with a requestor. The requestor retrieves the address of the endpoint from the ledger using the DID and requests access to the record from the wallet. The wallet verifies that the requestor has permission and provides access to the record.

### BACKGROUND

The storage and maintenance of health records for a patient is typically handled by a variety of medical providers that a patient has had a relationship with. For example, a patient may have a relationship with medical providers such as a dermatologist, a dentist, and an allergist and each provider may keep and maintain records regarding visits, medical procedures, and medications taken by the patient.

There are many drawbacks associated with the is approach. In particular, the patient is not fully in control of their own health records. For example, in the event that the patient would like to share their medical records with a new medical provider, the patient must contact each provider separately and facilitate the transfer of the records to the new medical provider.

### SUMMARY

In an embodiment, systems and methods for using a virtual health wallet are provided. The virtual health wallet is a data structure that is created for a patient by a self sovereign identity service and is associated with a decentralized digital identifier that is associated to the patient along with a public and private key pair. The decentralized digital identifier of the patient is stored on a distributed ledger. The wallet is stored on a cloud-based endpoint in a distributed manner. When an electronic medical record is created for a patient by a medical provider, the record is stored in the wallet at an endpoint. And a decentralized digital identifier of the medical record is stored in the distributed ledger along with an address of the end point. Later the user may share the decentralized digital identifier of the medical record with a third party. The third party retrieves the address of the end point from the distributed ledger using the decentralized digital identifier and requests access to the medical records from the wallet. The wallet verifies that the third party has permission, and if so, provides access to the medical record.

The systems and methods described herein provide at least the following advantages. First, because the electronic health records are stored in a distributed manner, the risk that the records of a patient are greatly reduced. Second, because all of the patient records are stored together in a digital wallet under the control of the patient, the patient can easily grant and revoke access to their medical records to interested third parties.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying figures, which are incorporated herein and form part of the specification, illustrate systems and methods for providing access to electronic health records using a virtual health wallet. Together with the description, the figures further serve to explain the principles of the systems and methods for providing access to electronic health records using a virtual health wallet described herein and thereby enable a person skilled in the pertinent art to make and use the systems and methods for providing access to electronic health records using a virtual health wallet.

FIG. 1 is an example environment for providing access to electronic health records using a virtual health wallet;

FIG. 2 is an illustration of a method for creating and using a virtual health wallet;

FIG. 3 is an illustration of a method for using a virtual health wallet;

FIG. 4 is an illustration of example electronic health records; and

FIG. 5 shows an example computing environment in which example embodiments and aspects may be implemented.

### DETAILED DESCRIPTION

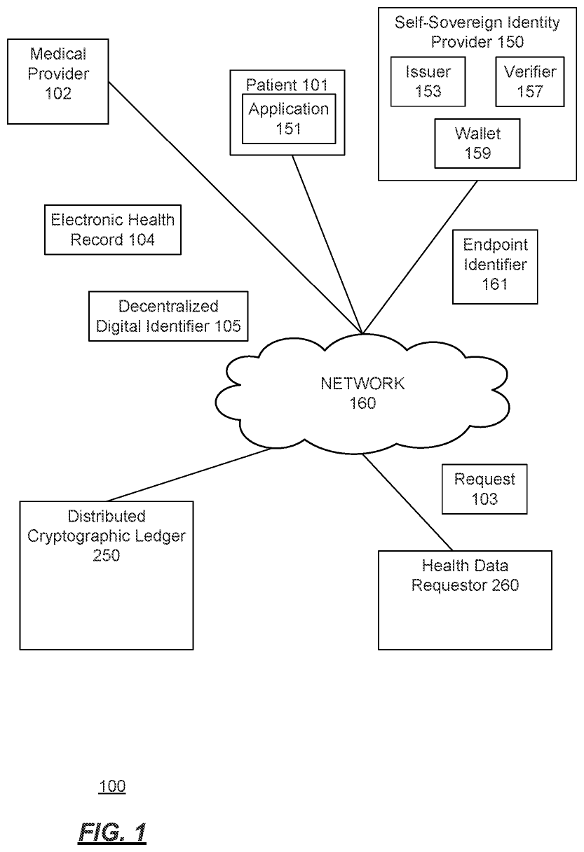

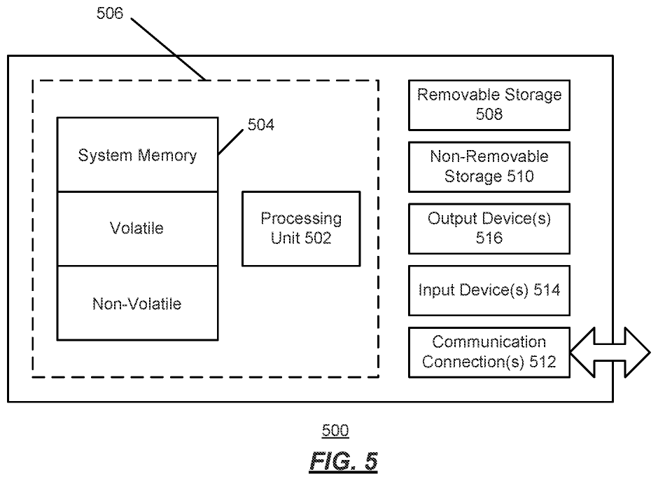

FIG. 1 is an example environment for providing access to electronic health records using a virtual health wallet. As shown, the environment 100 may include one or more medical providers 102, one or more self-sovereign identity (“SSI”) providers 150, one or more distributed cryptographic ledgers 250, one or more health data requestors 260, and one or more patients 101 in communication through a network 160. The network 160 may include a combination of private networks (e.g., LANs) and public networks (e.g., the Internet). Each of one or more medical providers 102, one or more SSI providers 150, one or more distributed cryptographic ledgers 250, one or more health data requestors 260, and one or more patients 101 may be partially implemented by one or more general purpose computing devices such as the computing device 500 illustrated in FIG. 5.

The SSI provider 150 may include an issuer 153 that issues one or more decentralized digital identifiers (“DID”) 105. Each DID 105 may uniquely identify an entity such as a medical provider 102, health data requestor 260, or patient 101. In addition, each DID 105 may uniquely identify a wallet 159 and an electronic health record 104.

Each DID 105 may be stored on the distributed cryptographic ledger 250 along with a corresponding DID document. The DID document may include information such an endpoint identifier 159 associated with the DID 105 and a public key associated with the entity associated with the DID 105.

The issuer 153 may create a virtual health wallet 159 for a patient 101. In some embodiments, the patient 101 may create a wallet by sending a request 103 to the issuer 153. In some embodiments, the request 103 may be sent by an application 151 executing on a computing device associated with the patient 101. The patient 101 may use the application 151 to manage their wallet 159, view their electronic health records 104 in their wallet 159, and to grant or deny access to the electronic health records 104 to one or more health data requestors 260.

In some embodiments, the issuer 153 may create a virtual wallet 159 for the patient 101 using the DID 105 associated with the patient 101. The virtual health wallet 159 may include a public and private key associated with the patient 101 and may initially include a node representing the patient 101. The node may be associated with the DID 105 of the patient 101.

After the creating the virtual wallet 159, the patient 101 may add an electronic health record 104 to their wallet 159. In some embodiments, the patient 101 may use the application 151 to send a request 103 to a medical provider 102 that generated the electronic health record 104. In response, the provider 102 may use the issuer 153 to create a DID 105 for the electronic health record 104 and may sign the electronic health record 104 using their private key. The medical provider 102 may then provide the signed electronic health record 104 to the SSI provider 150 who may store the electronic health record 104 at an endpoint associated with the wallet 159. The SSI provider 150 may then store the DID 105 for the electronic health record 104 on the distributed cryptographic ledger 250. The DID document associated with the stored DID 105 may include an endpoint identifier 161 that identifies the endpoint where the electronic health record is stored.

The medical provider 102 may also be associated with a DID 105. The DID 105 may be associated with the public key of the medical provider 102. The SSI provider 150 upon receiving the signed electronic health record 104 from the provider 102, may use the public key of the provider to authenticate the electronic health record 104. The SSI provider 150 may retrieve the public key of the provider 102 from the distributed cryptographic ledger 250 using the DID 105.

Later the patient 101 may desire to share an electronic health record 104 in their wallet 159 with a health data requestor 260. For example, the health data requestor 260 may be a new doctor that the patient 101 is visiting and would like to provide the doctor access to certain electronic health records 104.

In order to share the electronic health record 104 with the health data requestor 260, the patient 101 may provide the health requestor 260 the DID 105 associated with the electronic health record 104. In some embodiments, the patient 101 may use the application 151 to select the record 104 and the application 151 may provide the associated DID 105 to the health data requestor 260. The associated DID 105 may be provided in an electronic message such as an email. Alternatively, the application 151 may display a QR code with the DID 105 on a display of a smart phone of the patient 101 and the health data requestor 260 may scan the QR code to receive the DID 105. Other methods for sharing a DID 105 may be supported.

The health data requestor 260 may retrieve the DID document associated with the DID 105 from the distributed cryptographic ledger 250. The health data requestor 260 may retrieve the endpoint identifier 161 associated with the DID 105 from the DID document and may request the electronic health record 104 stored at the identified endpoint from the wallet 159.

In response to retrieving the request for the electronic health record 104, the verifier 157 may determine whether the health data requestor 260 has permission to access the electronic health record 104. Depending on the embodiment, the verifier 157 may maintain a list of the health data requestors 260 that have permission to access each electronic health record 104. The list may include the DID 105 associated with each health data requestor 260 that has permission to access the record. If the patient 101 later determines to revoke access from a health data requestor 260, the verifier 157 may remove the health data requestor 260 from the list.

If the health data requestor 260 has permission to view the record, then the verifier 157 may retrieve the electronic health record 104 from the associated endpoint and may provide the electronic health record 104 to the health data requestor 260. The electronic health record 104 may be signed using a private key of the wallet 159 and/or patient 101 so that the health data requestor 260 can verify that the electronic health record 104 was provided from the wallet 159. Because the electronic health record 104 was also signed by the private key of the medical provider 102, the health data requestor 260 can verify that the electronic health record 104 was in fact provided by the medical provider 102.

If the health data requestor 260 does not have permission to view the electronic health record 104, the verifier 157 may request permission from the patient 101. For example, the verifier 157 may cause the application 151 to prompt the patient 101 to provide or deny access to the electronic health record 104.

In some embodiments, the health data requestor 260 may have digitally signed the request 103 for the electronic health record 104. The verifier 157 may then verify that the request 103 comes from the health data requestor 260 using the public key of the health data requestor 260. The public key may be stored in the DID document associated with the DID 105 of the health data requestor 260 stored on the distributed cryptographic ledger 250.

In some embodiments, the electronic health records 104 may be stored in the wallet 159 as a directed acyclic graph. The directed graph may include a node for the patient and a child node for each different medical provider 102. Each medical provider node may be associated with a DID 105 and the public key of the associated medical provider 102. Each medical provider node may have child nodes that store electronic health records 104 containing health related data of the patient 101 and corresponding to medical procedures or medical services provided to the patient 101 by the medical provider 102. These record nodes may have child nodes that contain changes or updates made to the electronic health record 104 associated with the parent node.

As may be appreciated, a wallet may be used to store the electronic health records for multiple patients 101. For example, a patent 101 may use their electronic wallet to store electronic health records 104 for themselves and each of their dependent children. As another example, a patient 101 may use their electronic wallet to store electronic health records 104 for themselves and a dependent parent.

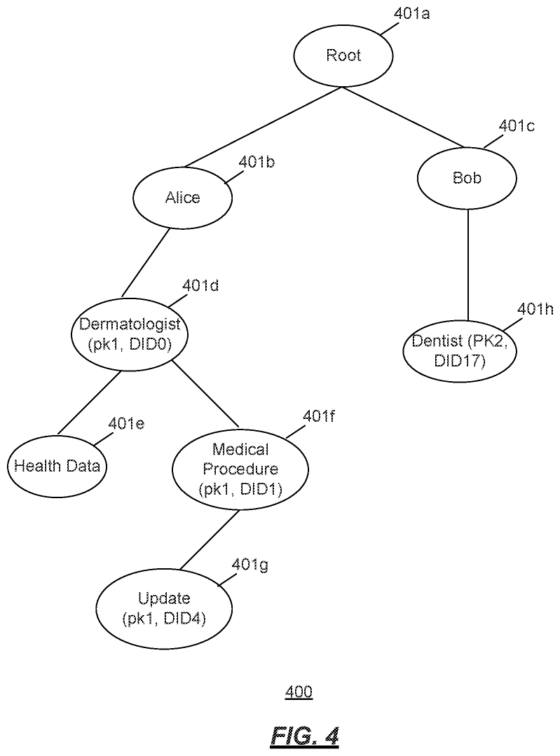

FIG. 4 is an illustration of an example directed acyclic graph 400 used to store electronic health records 104. In the example shown, the graph 400 holds the electronic health records 104 for two patients 101 named Alice and Bob. Bob may be a minor child of Alice.

The graph 400 includes a plurality of nodes. The root node 401a includes an edge directed to a sub-graph that includes the electronic health records 104 for Alice and an edge directed to a sub-graph that includes the electronic health records 104 for Bob.

The sub-graph for Allice starts with the node 410b and includes an edge to a node 401d representing a medical provider 102. As shown the sub-graph starting with node 401d is associated with a dermatologist and includes the public key of the dermatologist and a DID 105 (i.e., the DID0). The child nodes of the node 401d include the nodes 401e and 401f. The node 401e includes health data associated with Alice, and the node 401f includes information about a medical procedure performed on Alice by the dermatologist. The node 401f includes the public key of the dermatologist (i.e., pk1) and a DID 105 (i.e., DID1). The node 401f includes an edge to the node 401g that represents an update made to the electronic health record 104 associated with the medical procedure. The node 401g includes the public key of the dermatologist and a DID 105 (i.e., DID4).

The sub-graph for Bob starts with the node 410c and includes an edge to a node 401h representing a medical provider 102. As shown, the node 401h includes electronic health records 104 associated with a Dentist visit. The node 401h include the public key of the dentist (i.e., PK2) and a DID 105 (i.e., DID 17).

Returning to FIG. 1, when a patient 101 wishes to share an electronic health record 104, the patient 101 may provide the DID 105 corresponding to the root node of the subgraph that the patient 101 would like to provide access to. For example, referring to FIG. 4, the patient 101 may desire to share all of their dermatological records with a health data requestor 260. Accordingly, the patient 101 may share the DID 105 of the node 401d (i.e., DID0). Upon verifying the health data requestor 260, the verifier 157 may then return the electronic health records 104 stored at each of the endpoints associated with the DIDs 105 of the nodes 401d, 401e, 401f, and 401g.

In contrast, if the patient 101 wished to share only a particular electronic health record 104, the patient 101 may provide the DID 105 corresponding to only the particular electronic health record 104. For example, referring again to FIG. 4, the patient 101 may desire to share only the electronic health record 104 corresponding to a medical procedure with a health data requestor 260. Accordingly, the patient 101 may share the DID 105 of the node 401f (e.g., DID1). Upon verifying the health data requestor 260, the verifier 157 may then return the electronic health records 104 stored at each of the endpoints associated with the DIDs 105 of the nodes 401f and 401g.

The virtual health wallet 159 allows for the easy transfer of electronic health records 104 between wallets 159. For example, a first patient 101 may become a caregiver with respect to a second patent 101. So that the first patient 101 can share the electronic health records of the second patient 101, the first patient 101 may desire to transfer the electronic health records 104 of the second patient 101 from their wallet 159 to the wallet 159 of the second patient 101.

To facilitate such a transfer, the first patient 101 may receive access rights to the electronic health records 104 of the second patent 101 using a power of attorney or other legal procedure. The power of attorney may be stored by the first patient 101 or provided to an associated authorized entity (e.g., a government entity).

The first patient 101 may provide an identifier of the second patient 101 to the authorized entity along with a request 103 for their electronic health records 104. The identifier may a social security number. The authorized entity may use a mapping of social security numbers to DIDs 105 to determine the DID 105 of the electronic health records of the second patent 101.

The authorized entity may retrieve the endpoint identifier of the electronic health records 104 of the second patient 101 from the distributed cryptographic ledger 250. The authorized entity may determine that the first patient 101 has access rights to the health records 104, and may retrieve the electronic health records 104 from the identified endpoint (e.g., the wallet 159 of the second patient 101). The authorize entity may then sign the electronic health records 104 and may store them in an endpoint associated with the wallet 159 of the first patient 101. A new DID 105 may be assigned to the electronic health records 104, and an identifier of the endpoint associated with the wallet 159 may be stored on the distributed cryptographic ledger 250 with the new DID 105.

In another example, a first patient 101 may be the parent of a second patent 101. Once the second patient 101 turns 18 years old, they may desire to store their electronic health records 104 in their own wallet 159 rather than the wallet 159 of the first patient 101 (i.e., their parent).

First, the second patient 101 may create a wallet 159 on the SSI provider 150. The second patient 101 may then send a request 103 to the first patient 101 for their records 104. The first patient 101 may receive the request 103, and in response may instruct the SSI provider 150 to retrieve the requested electronic health records 104 from their wallet 159 and sign the record using their private key.

The SSI provider 150 may store the signed electronic health record at an endpoint associated with the wallet 159 of the second patient 101. The SSI provider 150 may assign a new DID 150 to the stored electronic health record 140, and may store the DID 150 and an identifier of the endpoint on the distributed cryptographic ledger 250.

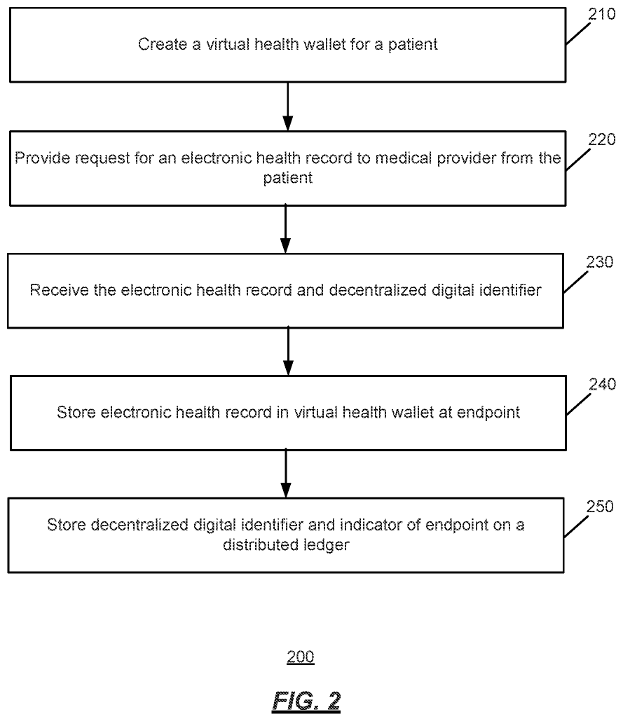

FIG. 2 is an illustration of a method for creating and using a virtual health wallet. The method 200 may be performed by the SSI provider 150.

At 210, a virtual health wallet is created. The virtual health wallet 159 may be created by the issuer 153 on behalf of a patient 101. The patient 101 may be associated with a DID 105 that uniquely identifies the patient 101. The DID 105 may be stored on a distributed cryptographic ledger 250 with a DID document that includes a public key of the patent 101. The patient 101 may request that the wallet 159 be created using an application 151 installed on a smart phone or other computing device.

At 220, a request for an electronic health record is provided to a medical provider. The request 103 may be provided by the patient 101 to a medical provider 102 that has an electronic health record 104 associated with the patient 101. The patient 101 may desire for the provider 102 to place the electronic health record 104 in the wallet 159. The request 103 may be signed using the private key of the patient 101 so the medical provider 102 can verify the patient 101 by retrieving the DID 105 of the patient 101 from the distributed cryptographic ledger 250.

At 230, the electronic health record is provided. The electronic health record may be provided by the medical provider 102 to the SSI provider 150. The electronic health record 104 may be signed using the private key of the medical provider 102. The electronic health record 104 may be associated with a DID 105. The DID 105 may be assigned by the issuer 153 of the SSI provider 150.

At 240, the electronic health record is stored in the virtual health wallet at an endpoint. The endpoint may be an SSI service endpoint. The electronic health record may be stored in a directed acyclic graph.

At 250, the DID and an endpoint identifier are stored. The DID 105 and the endpoint identifier 159 may be stored by the SSI provider 150 in the distributed cryptographic ledger 250. The endpoint identifier 159 may be stored in a DID document stored on the distributed cryptographic ledger 250 with the DID 105.

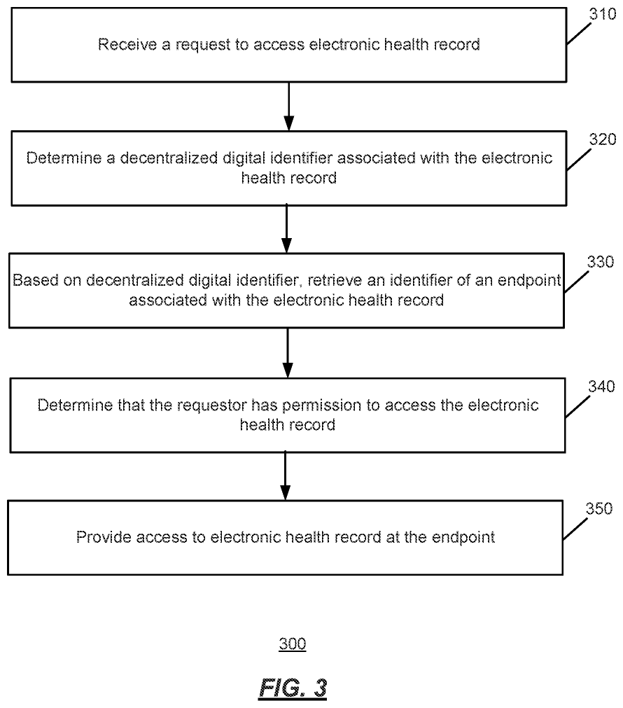

FIG. 3 is an illustration of a method for using a virtual health wallet. The method 300 may be implemented by the SSI provider 150.

At 310, a request to access an electronic health record is received. The request 103 may be received by the SSI provider 150 from a health data requestor 260. The requestor 260 may desire to view an electronic health record 104 stored in a wallet 159 associated with a patient 101. In some embodiments, the request 103 may be signed by a private key associated with the health data requestor 260. The SSI provider 150 may verify the health data requestor 260 using a DID 105 associated with the health data requestor 260 and the digital signature.

At 320, a DID associated with the electronic health record is determined. The DID 105 may be determined by the verifier 157. The DID 105 may be provided in the request 103 to access the electronic health record 104.

At 330, based on the DID, an identifier of an endpoint associated with the electronic health record is determined. The endpoint identifier 159 may be determined by the verifier 157. In some embodiments, the verifier 157 may retrieve the DID document corresponding to the DID 105 from the distributed cryptographic ledger 250. The endpoint identifier 159 may be then extracted from the DID document.

At 340, that the requestor has permission to access the electronic health record is determined. The determination may be made by the verifier 157. Depending on the embodiment, the verifier 157 may check a list of health data requestors 260 authorized to access the electronic health record 104 associated with the DID 105. If the DID 105 is not on the list, the verifier 157 may ask the patient 101 for permission via the application 151.

At 350, access to the electronic health record is provided. The access may be provided to the health data requestor 260 by the SSI provider 150. Depending on the embodiment, the health data requestor 260 may be provided access to the electronic health record 104 at the endpoint or the electronic health record 104 may be provided or transmitted to the health data requestor 260.

FIG. 5 shows an example computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 5, an example system for implementing aspects described herein includes a computing device, such as computing device 500. In its most basic configuration, computing device 500 typically includes at least one processing unit 502 and memory 504. Depending on the exact configuration and type of computing device, memory 504 may be volatile (such as random access memory (RAM)), non-volatile (such as read-only memory (ROM), flash memory, etc.), or some combination of the two. This most basic configuration is illustrated in FIG. 5 by dashed line 506.

Computing device 500 may have additional features/functionality. For example, computing device 500 may include additional storage (removable and/or non-removable) including, but not limited to, magnetic or optical disks or tape. Such additional storage is illustrated in FIG. 5 by removable storage 508 and non-removable storage 510.

Computing device 500 typically includes a variety of computer readable media. Computer readable media can be any available media that can be accessed by the device 500 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 504, removable storage 508, and non-removable storage 510 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 500. Any such computer storage media may be part of computing device 500.

Computing device 500 may contain communication connection(s) 512 that allow the device to communicate with other devices. Computing device 500 may also have input device(s) 514 such as a keyboard, mouse, pen, voice input device, touch input device, etc. Output device(s) 516 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although example implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A method comprising:
creating a virtual health wallet by a computing device, wherein the virtual health wallet is associated with a patient and includes a public key and a private key;
providing a first request for an electronic health record to a medical provider from the patient by the computing device;
receiving the electronic health record from the medical provider by the computing device, wherein the electronic health record is associated with a first decentralized digital identifier;
storing the electronic health record in the virtual health wallet by the computing device, wherein the electronic health record is associated with a first endpoint of the virtual health wallet; and
storing the first decentralized digital identifier for the electronic health record with an identifier of the first endpoint associated with the electronic health record on a distributed cryptographic ledger by the computing device.

2. The method of claim 1, wherein storing the electronic health record in the virtual health wallet comprises:
creating a first node for the electronic health record by the computing device, wherein the first node comprises the electronic health record and the first decentralized digital identifier.

3. The method of claim 2, further comprising:
receiving an update to the electronic health record from the medical provider by the computing device, wherein the update to the electronic health record is associated with a second decentralized digital identifier; and
storing the updated electronic health record in the virtual health wallet by the computing device, wherein the electronic health record is associated with a second endpoint of the virtual health wallet.

4. The method of claim 3, wherein storing the updated electronic health record in the virtual health wallet comprises:
creating a second node for the updated electronic health record by the computing device, wherein the second node comprises the update to the electronic health record and the second decentralized digital identifier; and
adding a link to the second node to the first node.

5. The method of claim 1, further comprising:
receiving a second request to access the electronic health record, wherein the second request includes the first decentralized digital identifier and is associated with a second medical provider;
in response to the second request, retrieving the identifier of the first endpoint from the distributed cryptographic ledger;
determining that the second medical provider has permission to access the electronic health record; and
in response to determining that the second medical provider has permission, providing access to the electronic health record on the first endpoint.

6. The method of claim 1, wherein storing the electronic health record in the virtual health wallet comprises signing the electronic health record with a private key of the medical provider.

7. The method of claim 5, wherein determining that the second medical provider has permission to access the electronic health record comprises:
prompting the patient for permission using an application executing on a computing device associated with the patient.

8. The method of claim 5, wherein the second request is associated with a digital signature and further comprising authenticating the second request using a public key associated with the second medical provider.

9. A method comprising:
receiving a request to access an electronic health record stored on a virtual health wallet for a patient by a computing device, wherein request is received from a first medical provider;
in response to the request, determining a decentralized digital identifier associated with the electronic health record by the computing device;
based on the decentralized digital identifier, retrieving an identifier of an endpoint associated with the electronic health record by the computing device;
determining that the first medical provider has permission to access the electronic health record by the computing device; and
in response to determining that the first medical provider has permission, providing access to the electronic health record at the endpoint.

10. The method of claim 9, wherein retrieving the identifier of the endpoint associated with the electronic health record comprises retrieving the identifier of the endpoint from a distributed cryptographic ledger using the decentralized digital identifier.

11. The method of claim 9, wherein the decentralized digital identifier is received from the patient.

12. The method of claim 11, wherein the decentralized digital identifier is received from the patient via a QR code displayed on a smart phone associated with the patient.

13. The method of claim 9, wherein determining that the first medical provider has permission to access the electronic health record comprises requesting permission from the patient through an application installed on a smart phone associated with the patient.

14. The method of claim 9, further comprising:
creating the virtual health wallet;
providing a second request for the electronic health record to a second medical provider from the patient by the computing device;
receiving the electronic health record from the second medical provider, wherein the electronic health record is associated with the decentralized digital identifier;
storing the electronic health record in the virtual health wallet, wherein the electronic health record is associated with the endpoint of the virtual health wallet; and
storing the decentralized digital identifier for the electronic health record with an identifier of the endpoint associated with the electronic health record on the distributed cryptographic ledger.

15. A system comprising:
at least one processor; and
a computer-readable medium storing computer-executable instructions that when executed by the at least one processor cause the system to:
create a virtual health wallet, wherein the virtual health wallet is associated with a patient and includes a public key and a private key;
provide a first request for an electronic health record to a medical provider from the patient;
receive the electronic health record from the medical provider, wherein the electronic health record is associated with a first decentralized digital identifier;
store the electronic health record in the virtual health wallet, wherein the electronic health record is associated with a first endpoint of the virtual health wallet; and
store the first decentralized digital identifier for the electronic health record with an identifier of the first endpoint associated with the electronic health record on a distributed cryptographic ledger.

16. The system of claim 15, wherein storing the electronic health record in the virtual health wallet comprises:
creating a first node for the electronic health record, wherein the first node comprises the electronic health record and the first decentralized digital identifier.

17. The system of claim 16, further comprising:
receiving an update to the electronic health record from the medical provider, wherein the update to the electronic health record is associated with a second decentralized digital identifier; and
storing the updated electronic health record in the virtual health wallet, wherein the electronic health record is associated with a second endpoint of the virtual health wallet.

18. The system of claim 17, wherein storing the updated electronic health record in the virtual health wallet comprises:
creating a second node for the updated electronic health record, wherein the second node comprises the update to the electronic health record and the second decentralized digital identifier; and
adding a link to the second node to the first node.

19. The system of claim 15, further comprising:
receiving a second request to access the electronic health record, wherein the second request includes the first decentralized digital identifier and is associated with a second medical provider;
in response to the request, retrieving the identifier of the first endpoint from the distributed cryptographic ledger;
determining that the second medical provider has permission to access the electronic health record; and
in response to determining that the second medical provider has permission, providing access to the electronic health record on the first endpoint.

20. The system of claim 15, wherein storing the electronic health record in the virtual health wallet comprises signing the electronic health record with a private key of the medical provider.
